(19) United States
(12) Patent Application Publication
(10) Pub. No.: US 2008/0286384 A1
(43) Pub. Date: Nov. 20, 2008

(54) Topical Wound Care Product Containing Hyssop

(76) Inventor: Renee Reichert, Romeoville, IL (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 11/575,380

(22) PCT Filed: Sep. 15, 2004
(86) PCT No.: PCT/US04/29930

§ 371 Date: Oct. 31, 2007

(51) Int. Cl. A61K 36/53; A61P 17/02

(52) U.S. Cl. 424/725

(57) ABSTRACT

A composition for treating wounds having an effective amount of hyssop extract, combined with glycerol, salt, saccharides, and water has been discovered to be effective in promoting healing of wounds.

### BACKGROUND

This invention relates to a topical wound care product comprised of a liquid extract of hyssop and a method of treating wounds with this product.

### BRIEF SUMMARY OF THE INVENTION

A composition for treating wounds having an effective amount of hyssop extract, combined with glycerol, salt, saccharides, and water has been discovered to be effective in promoting healing of wounds.

### DETAILED DESCRIPTION

A variety of hyssop-containing compositions can be used to topically treat wounds, especially difficult healing wounds such as those seen in diabetics. The preferred composition comprises:

60% by volume of a hyssop extract (described below)

5% glycerol by volume

5% sodium chloride by weight (preferably rock salt) or volume

15% rock sugar by weight

sufficient sterile water to bring to volume

Thus, to make 100 milliliters of the composition, 60 mls of an aqueous hyssop extract would be combined with 5 mls glycerol, and 5 grams sodium chloride and 15 grams of sugar would be added. Then, sterile water would be added to bring the composition to 100 mls final volume.

After combining these ingredients, the composition is heated to at least 220° F., but not more than 375° F., with the temperature maintained for 10 to 15 minutes until the ingredients are completely dissolved. At present, the composition is heated in a Dutch oven on a cooktop and, as necessary, the temperature is monitored with a high temperature thermometer. If the temperature is too high, then the composition boils off and if the composition does not reach the minimum temperature, a sticky precipitate is formed.

The composition is then strained four times with a fine mesh kitchen strainer and then a fifth time with cheese cloth. The resulting composition is a greenish brown liquid, having a viscosity between water and maple syrup.

Without allowing the strained composition to reach room temperature, it is then optionally further heated over low heat (about 200° F.) for about a half hour, and then gradually cooled to room temperature (about 78° F.). The composition is then covered and rests for at least two hours at room temperature and preferably no more than four hours, before it is transferred into amber bottles for storage at room temperature until use. If there is precipitate, the composition should be strained.

One source of an acceptable hyssop extract is the Nutritional Network Associates, 326 East 75^{th} Street, Chicago, Ill. 60619, USA. The extract's label indicates it is distributed by TNT PLUS DYN-O-VITE Inc., 1925 East 59th Street, Cleveland, Ohio 44106. This extract is described as a proprietary mixture of hyssop, comprising fresh hyssop extract (aerial parts) (also known as the Holy Herb and Hyssopus officinalis), water, ethanol (less than 5%) and glycerol (glycerine USP). The extract's labelling indicates that this proprietary mixture is present in 5 g per teaspoon, indicating that no other ingredients are present in this extract. It is believed that this extract is about 60% hyssop. The extract is provided in amber bottles.

Another acceptable source of hyssop extract is Nature's Answer Hyssop Alcohol Free Extract NA-00638, available from www.totaldiscountvitamins.com, 170 Fulton Street, Farmingdale, N.Y. 11735. This extract is described as containing hyssop herb fluid extract (1:1) 2000 mg. in 2 ml.

The hyssop extract may be made from dried hyssop leaves. In one embodiment, the dried leaves were brought to a boil and then simmered uncovered, in an equal volume of water, for about 30 minutes, resulting in a tea-like liquid. The liquid was strained through cheesecloth, forming a hyssop extract. Sugar (½ volume of the initial volume of leaves) and salt (2 volumes of the initial volume of leaves) were added, forming a pasty preparation which was effective.

Alternate methods of extraction, including filtration with water or glycerol, may work. The hyssop extract is expected to be effective even if the hyssop concentration is as low as 6% by volume in the final composition. Thus, 60 mls of a 10% pure hyssop extract in 100 mls is expected to be effective.

The glycerol in the composition is used to provide a moisture barrier, which keeps the wound moist and allows granular tissue to form instead of the less-desired fibronic tissue.

The sterile water may be from any source. It is believed that deionized and distilled water work equally well, as does local tap water (from Lake Michigan). The water serves two purposes: to allow the composition to be readily applied and to keep the wound moist during healing.

The sodium chloride acts as an osmotic, and helps to retain moisture in the healing area. Although rock salt is preferred, any sodium chloride product is expected to work, including 0.45% saline solutions. If a 0.45% saline solution is used, 5 mls of the saline solution is used in place of the 5 grams of rock salt when making 100 milliliters of the composition, resulting in a final concentration of about 0.0225% salt. Thus, a wide range of sodium chloride concentrations is suitable. Epsom salts (magnesium sulfate) also work at similar concentrations.

The sugar is believed to act as a carbon source and attract bacteria to the sugar, and away from the healing tissue. Thus, any saccharide, whether a monosaccharide, a disaccharide, or a polysaccharide, including cellulose, starch, sucrose, sorbitol, maltose, lactose, fructose, and glucose, and the like, would be expected to be acceptable. In the preferred embodiment, rock sugar (purchased from a candy store) is used because it is believed that it goes into solution more readily and, if any should not go into solution, the resulting precipitate is larger granules and more readily filtered out. (Granular sugar tends to reprecipitate and plug the pump if it is applied using a sprayer.)

A variety of sucrose compositions are believed to be acceptable, including granulated sugars, brown sugars, and liquid sugars. Honey, molasses, and other sugar-containing products are also expected to be acceptable.

I have found that a paste-like composition using additional sugar, up to at least 25 grams (with a concomitant reduction in water) per 100 mls, also heals wounds. Likewise, using petroleum jelly as a thickening agent worked in initial studies. I believe that other thickening agents, including talc and corn starch, would provide acceptable results.

The composition is applied to wounds in a variety of ways. It may be sprayed on via an atomizer, or injected, or painted, or via dropper, or using a saturated gauze or sponge for packing the wound, or in a moisture-retaining package, or as a cream or paste.

In treating wounds, I generally use the following protocol:

If the wound type is non-healing, typically featuring fibrous infected tissue, I first do a sharp debridement (e.g., I use a scalpel or the like to clean the wound). If the product described herein is not used, such a sharp debridement must be done every three to five days, resulting in great discomfort to the patient and increased medical costs. If the hyssop-containing product is used, then sharp debridement is decreased to every 14-21 days and, often, is not required at all after the initial debridement.

As necessary, I also manually debride the wound by using a gauze pad or sterile washing techniques or the like in order to clean up the wound. After the wound has been washed out (with water and optionally soap) I rinse it with saline or commercially available cleaners such as Wound Cleanze.

The product is then applied, preferably via a pump spray or eye dropper. If the wound is deep, the product may be injected with a syringe. An antibiotic may be used in combination with applying the present product if the wound is infected. Once the product is applied, the wound is covered with sterile dressings. Preferably, the cover is non-occlusive because it is believed that it is important that the wound be exposed to oxygen to enable the composition to work. An occlusive cover is not preferred because drainage is inhibited. However, the product has been successfully used to treat an abdominal wound of such size that the patient was enveloped in plastic wrap during healing. It may be that such a wrapping was not occlusive, especially since substantial drainage did result.

Preferably, the product is reapplied, preferably approximately every 24-72 hours until healing is satisfactorily complete. More frequent application than every twelve hours is not desired because it is believed that the product must be in contact with the wound for sufficient time to induce a hyperemic response. Although it is believed that longer than 72 hours would work, in practice, the product (as described above) has been absorbed and the dressing is dry. If the product were more paste-like, a longer time between reapplication would be possible.

I have observed that using the composition results in significant healing response. A significant healing response includes decrease in wound size, proliferation of granulation tissue, absence of heavy or foul discharge, and absence of progressive or recurring necrosis. Specifically, I have observed 96 patients with wounds that had failed to heal by other wound care techniques, including patients with foot and ankle neuropathic wounds, arterial wounds, traumatic wounds, burn wounds, and pressure wounds. Many of the patients were diabetic, and all wounds had been present for at least four weeks. In all instances, at least a slight improvement based on wound size and appearance was seen when the product was applied. Eighty-four of the patients healed without further intervention over the next 14 to 90 days and 10 more healed to the point of being able to be closed surgically. The eighty-four patients had an average time to heal of thirty-six days (range of 14 to 90 days). Two patients remained unhealed. These two patients had arterial insufficiency and numerous co-morbidities, and one later had a lower extremity amputated and then died.

Specifically, we have seen a hyperemic response, with effective angiogenesis across the wound. Thus, new capillaries form a matrix across the wound and skin is formed therefrom. It appears that this product is an autolytic debritory product, as evidenced by the considerably reduced number of sharp debridements necessary to accomplish wound healing. This product is easy to handle, and no special skills are needed (once the wound is clean) to apply the product to a wound. It is believed that one cannot overdose with this product.

1. A topical wound treatment composition having an effective wound-treatment amount of hyssop, comprising: (a) from about 10% to about 95% hyssop extract by volume; (b) from about 0% to about 6% glycerol by volume; (c) from about 0% to about 5% salt by weight; and (d) from about 0 to about 30% saccharides by weight; and (d) from about 0% to about 15% water.

2. The composition of claim 1 wherein the hyssop extract is between about 50% and about 65%.

3. The composition of claim 1 wherein the glycerol is between about 4% and about 6%.

4. The composition of claim 1 wherein the salt is selected from the group consisting of sodium chloride and magnesium sulfate.

5. The composition of claim 4 wherein the salt is sodium chloride.

6. The composition of claim 5 wherein the sodium chloride is selected from the group consisting of rock salt, kosher salt, saline solutions, sea salt, table salt, and popcorn salt.

7. The composition of claim 5 wherein the sodium chloride is about 0.2% to about 5% by weight.

8. The composition of claim 6 wherein the sodium chloride is about 5% by weight.

9. The composition of claim 1 wherein the saccharides are between 0% and 15% by weight.

10. The composition of claim 9 wherein the saccharides are selected from the group consisting of monosaccharides, disaccharides, and polysaccharides.

11. The composition of claim 10 wherein the saccharides are selected from the group consisting of cellulose, starch, sucrose, sorbitol, maltose, lactose, fructose, and glucose.

12. The composition of claim 11 wherein the saccharide is sucrose.

13. The composition of claim 12 wherein the sucrose is selected from the group consisting of granulated sugars, brown sugars, and liquid sugars.

14. The composition of claim 13, wherein the sucrose is rock sugar.

15. The composition of claim 14, wherein the rock sugar is 15% by weight of the composition.

16. The composition of claim 1 comprising:
60% by volume of at least 10% pure hyssop extract
5% glycerol by volume
5% sucrose by weight,
15% sugar by weight, and
sufficient water to bring the composition to a chosen volume.

17. A topical wound treatment composition comprising:
60% by volume of at least 10% pure hyssop extract,
5% glycerol by volume,
5% sodium chloride by weight,
15% rock sugar by weight, and
sufficient water to bring the composition to a chosen volume.

18. A method of treatment of wounds in mammals, which method comprises administering an effective amount of hyssop in a topical composition to the wound.

19. The method of claim 18 wherein the topical composition comprises hyssop extract, glycerol, a salt, and a sugar.

20. The method of claim 19 wherein the topical composition comprises hyssop extract, glycerol, sodium chloride, and sucrose.

21. The method of claim 19 wherein the topical composition comprises:
60% by volume of at least 10% pure hyssop extract
5% glycerol by volume,
5% sodium chloride by weight and
15% rock sugar by weight, and
sufficient water to bring the composition to a chosen volume.
